(19) United States
(12) Patent Application Publication
(10) Pub. No.: US 2022/0168293 A1
(43) Pub. Date: Jun. 2, 2022

(54) TIME TO RESOLUTION OF AXITINIB-RELATED ADVERSE EVENTS

(71) Applicant: PFIZER INC., New York, NY (US)

(72) Inventor: Despoina Thomaidou, Athens (GR)

(73) Assignee: PFIZER INC., New York, NY (US)

(21) Appl. No.: 17/538,919

(22) Filed: Nov. 30, 2021

Related U.S. Application Data
(60) Provisional application No. 63/120,660, filed on Dec. 2, 2020.
(60) Provisional application No. 63/190,195, filed on May 18, 2021.
(60) Provisional application No. 63/245,439, filed on Sep. 17, 2021.
(60) Provisional application No. 63/275,764, filed on Nov. 4, 2021.

(51) Int. Cl. A61K 31/4439; C07K 16/28; A61P 35/00

(52) U.S. Cl. CPC A61K 31/4439; A61P 35/00; C07K 16/2827; C07K 16/2818

(57) ABSTRACT

This invention relates to a method of managing an adverse event in a renal cell carcinoma (RCC) patient undergoing treatment with axitinib, or a pharmaceutically acceptable salt thereof, wherein said method comprises interrupting axitinib, or a pharmaceutically acceptable salt thereof, treatment for at least 1-7 days to allow the adverse event to resolve before restarting treatment. Additionally, the invention relates to a method of managing an adverse event in an RCC patient undergoing treatment with a combination of axitinib, or a pharmaceutically acceptable salt thereof, and an immune-oncology (IO) agent, wherein said method comprises interrupting axitinib, or a pharmaceutically acceptable salt thereof, treatment for at least 4-11 days to allow the adverse event to resolve before restarting axitinib, or a pharmaceutically acceptable salt thereof, treatment.

### CROSS-REFERENCE TO RELATED APPLICATIONS

This application claims the benefit of U.S. Provisional Application No. 63/120,660 filed Dec. 2, 2020, U.S. Provisional Application No. 63/190,195 filed May 18, 2021, U.S. Provisional Application No. 63/245,439 filed Sep. 17, 2021 and U.S. Provisional Application No. 63/275,764 filed Nov. 4, 2021, the contents of which are hereby incorporated by reference in their entireties.

### FIELD OF THE INVENTION

The present invention relates to guidance for managing adverse events occurring in renal cell carcinoma patients being treated with axitinib alone or in combination with immune-oncology therapies, e.g., avelumab or pembrolizumab. The invention also relates to associated methods of treatment.

### BACKGROUND OF THE INVENTION

Recently, a new treatment paradigm for advanced renal cell carcinoma (aRCC) has been adopted which involves administration of a tyrosine kinase inhibitor (TKI) combined with an immune-oncology (IO) therapy, i.e., a programmed cell death 1 (PD-1) or a programmed cell death ligand 1 (PD-L1) antagonist antibody (National Comprehensive Cancer Network®, NCCN Clinical Practice Guidelines in Oncology (NCCN Guidelines®), Kidney Cancer version 2.2020; ESMO Guidelines Committee, Escudier B, Porta C, et al. eUpdate—renal cell carcinoma treatment recommendations 2, last update Feb. 26, 2020; Rini et al., J Immunother Cancer 7: 354, 2019; Monteiro et al., Clin Genitourin Cancer 18: 244-251, 2020; Alam et al., Am J Clin Oncol 43: 477-83, 2020). Axitinib, a TKI and a potent, selective inhibitor of vascular endothelial growth factor receptors (VEGFRs) (Bellesoeur et al., Drug Des Devel Ther 11: 2801-2811, 2017), is approved for the first-line treatment of patients with aRCC in combination with either the IO antibody therapy avelumab or pembrolizumab, and as a single agent, second-line treatment for the treatment of aRRC after failure of one prior systemic therapy. One area of concern for combined VEGFR TKI plus IO therapy is the possible occurrence of overlapping toxicities. Combinations of approved doses of the TKIs sunitinib or pazopanib together with anti-PD-1 agents such as nivolumab have proved to be very toxic, likely due to overlapping toxicities including fatigue and hepatic toxicity (Amin et al., J Immunother Cancer 6: 109, 2018). Combination studies involving other TKIs such as cabozantinib or lenvantinib and anti-PD-1 therapies are in progress (Mol Cancer Ther 18: 2185-93, 2019; Lee et al., J Clin Oncol 38: 5008, 2020), and the first results of CheckMate 9ER study of cabozantinib plus nivolumab were recently reported (Choueiri et al., Ann Oncol 31: S1142-51215, 2020). The half-lives of the various TKIs differ greatly, and this might influence the duration of the toxicities observed in these combination regimens.

If there is an occurrence of an adverse event (AE) with combined axitinib and IO therapy, early identification of the underlying etiology of the toxicity, i.e. axitinib- or immune-related, is important for the quick implementation of an appropriate management strategy (Rini et al., 2020, supra, Grunwald et al., Br J Cancer 123: 898-204, 2020). In the absence of serious clinical signs of immune-related toxicity, a potential first step in patient management is the interruption of axitinib treatment and observation for resolution or improvement (Rini et al., 2020, supra, Grunwald et al., 2020, supra). If the AE resolves or improves quickly, it is most likely due to axitinib toxicity. But, if it persists, then this could be a sign that it is immune-related and requires treatment with steroids. Assessing the time to resolution (TTR) of commonly reported AEs associated with axitinib treatment interruption or discontinuation, either as monotherapy or in combination with IO therapy, would provide evidence to support a management strategy for such patients. The aim of this study was to determine the TTR of common AEs after treatment interruption or discontinuation of axitinib used as monotherapy or in combination with IO therapy, and to compare these findings with the TTR of other TKIs.

As noted above, IO agents work as checkpoint inhibitors. Immune checkpoint proteins are found on the surface of T-cells, act as regulators of the immune system, and, in normal circumstances, prevent the immune system from attacking the body's own cells indiscriminately. However, tumors can leverage certain immune checkpoint pathways as a mechanism to evade an immune response. Inhibiting these checkpoint inhibitors with one or more IO agent in a cancer patient can enable the patient's own immune system to attack cancer cells.

IO agents include PD-1 and PD-L1 antagonist antibodies. The inhibition of PD-1 axis signaling through its direct ligands (e.g., PD-L1, PD-L2) has been proposed as a means to enhance T cell immunity for the treatment of cancer (e.g., tumor immunity). Moreover, similar enhancements to T cell immunity have been observed by inhibiting the binding of PD-L1 to the binding partner B7-1 (Ribas A. and Wolchok J., Science 359: 1350-1355, 2018).

PD-L1 is a cell-surface protein and member of the B7 family. PD-L1 is found on almost all types of lymphohematopoietic cells and is is expressed at low levels by resting T cells, B cells, macrophages and dendritic cells and is further up regulated by and anti-CD40 antibody for B cells, anti-CD3 antibody for T cells, anti-CD40 antibody, IFNγ and granulocyte macrophage colony-stimulating factor (GM-CSF) for macrophages and/or anti-CD40 antibody, IFNγ, IL-4, IL-12 and GM-CSF for Dendritic cells (DCs). PD-L1 is also expressed by some non-hematopoietic cells and is overexpressed in many cancers, wherein its overexpression is often associated with poor prognosis (Okazaki T et al., Intern. Immun. 2007 19(7):813, 2007) (Thompson R H et al., Cancer Res 66(7):3381, 2006). Interestingly, the majority of tumor infiltrating T lymphocytes predominantly express PD-1, in contrast to T lymphocytes in normal tissues and peripheral blood. PD-1 on tumor-reactive T cells can contribute to impaired antitumor immune responses (Ahmadzadeh et al., Blood 114(8): 1537, 2009). This may be due to exploitation of PD-L1 signaling mediated by PD-L1 expressing tumor cells interacting with PD-1 expressing T cells to result in attenuation of T cell activation and evasion of immune surveillance (Sharpe et al., Nat Rev 2002) (Keir M E et al., 2008 Annu. Rev. Immunol. 26:677, 2008). Therefore, inhibition of the PD-L1/PD-1 interaction may enhance T cell-mediated killing of tumors.

The other known ligand for PD-1, PD-L2, also known as B7-DC, Btdc, and CD273, is a cell surface protein. PD-L2 is expressed by antigen presenting cells, including dendritic cells, with expression also found in other non-hematopoietic tissues.

### SUMMARY OF THE INVENTION

The invention herein provides a method of managing an adverse event in a renal cell carcinoma (RCC) patient undergoing treatment with axitinib, or a pharmaceutically acceptable salt thereof, wherein said method comprises interrupting axitinib, or a pharmaceutically acceptable salt thereof, treatment to allow the adverse event to resolve before restarting treatment.

The invention herein provides a method of managing an adverse event in a renal cell carcinoma (RCC) patient undergoing treatment with axitinib, or a pharmaceutically acceptable salt thereof, wherein said method comprises interrupting axitinib, or a pharmaceutically acceptable salt thereof, treatment for at least 1-7 days to allow the adverse event to resolve before restarting treatment.

In some embodiments, the adverse event is diarrhea, hypertension, nausea, or palmar-plantar erythrodysesthesia syndrome. In some embodiments, the preferred duration of interruption of treatment is (or preferably, the treatment is interrupted for) at least 1, 2, 3, 4, 5, 6 or 7 days. In other embodiments, the duration of interruption is (or the treatment is interrupted for) 1-2, 1-3, 1-4, 1-5, 1-6, 1-7, 2-3, 2-4, 2-5, 2-6, 2-7, 3-4, 3-5, 3-6, 3-7, 4-5, 4-6, 4-7, 5-6, 5-7 or 6-7 days. In some embodiments, the duration of interruption of treatment is (or the treatment is interrupted) for 1-3 days. In some embodiments, the adverse event is Grade adverse event. In some embodiments, the adverse event is Grade adverse event and the duration of interruption of treatment is (or the treatment is interrupted) for 2-4 days.

In other preferred embodiments, the adverse event is fatigue and said method comprises interrupting treatment for at least 4-16 days. In some embodiments, the preferred interruption of treatment is at least 4, 5, 6, 7, 8, 9, 10, 11, 12, 13, 14, 15, or 16 days. In some embodiments, the duration of interruption of treatment is (or the treatment is interrupted) for 8 days. In some embodiments, the duration of interruption is (or the treatment is interrupted for) 4-5, 4-6, 4-7, 4-8, 4-9, 4-10, 4-11, 4-12, 4-13, 4-14, 4-15, 4-16, 5-6, 6-7, 5-8, 5-9, 5-10, 5-11, 5-12, 5-13, 5-14, 5-15, 5-16, 6-7, 6-8, 6-9, 6-10, 6-11, 6-12, 6-13, 6-14, 6-15, 6-16, 7-8, 7-9, 7-10, 7-11, 7-12, 7-13, 7-14, 7-15, 7-16, 8-9, 8-10, 8-11, 8-12, 8-13, 8-14, 8-15, 8-16, 9-10, 9-11, 9-12, 9-13, 9-14, 9-15, 9-16, 10-11, 10-12, 10-13, 10-14, 10-15, 10-16, 11-12, 11-13, 11-14, 11-15, 11-16, 12-13, 12-14, 12-15, 12-16, 13-14, 13-15, 13-16, 14-15, 14-16, or 15-16 days.

In another embodiment, the invention provides a method of managing an adverse event in an RCC patient undergoing treatment with a combination of axitinib, or a pharmaceutically acceptable salt thereof, and an immune-oncology (IO) agent, wherein said method comprises interrupting axitinib, or a pharmaceutically acceptable salt thereof, treatment to allow the adverse event to resolve before restarting axitinib, or a pharmaceutically acceptable salt thereof, treatment.

In another embodiment, the invention provides a method of managing an adverse event in an RCC patient undergoing treatment with a combination of axitinib, or a pharmaceutically acceptable salt thereof, and an immune-oncology (IO) agent, wherein said method comprises interrupting axitinib, or a pharmaceutically acceptable salt thereof, treatment for at least 4-11 days to allow the adverse event to resolve before restarting axitinib, or a pharmaceutically acceptable salt thereof, treatment.

In some such embodiments, the adverse event is diarrhea, fatigue, hypertension, nausea, or palmar-plantar erythrodysthesia. In some embodiments, the duration of interruption is (or the treatment is interrupted for) 4-5, 4-6, 4-7, 4-8, 4-9, 4-10, 4-11, 5-6, 5-7, 5-8, 5-9, 5-10, 5-11, 6-7, 6-8, 6-9, 6-10, 6-11, 7-8, 7-9, 7-10, 7-11, 8-9, 8-10, 8-11, 9-10, 9-11, or 10-11 days. In some embodiments, the adverse event is Grade adverse event.

In other embodiments, the IO agent is a programmed cell death protein 1 (PD-1) antagonist or a programmed cell death ligand 1 (PD-L1) antagonist.

In some preferred embodiments, the IO agent is pembrolizumab or avelumab.

The invention also contemplates embodiments where the method further comprises considering reducing the dose of axitinib, or a pharmaceutically acceptable salt thereof, when restarting treatment as per recommended dose modification guidelines.

In another embodiment, the invention comprises reducing the dose of axitinib, or pharmaceutically acceptable salt thereof, when restarting treatment as per recommended dose modification guidelines.

For any embodiment where the RCC patient is undergoing treatment with axitinib, or a pharmaceutically acceptable salt thereof, and an IO agent, another embodiment further comprises reducing the dose of axitinib, or a pharmaceutically acceptable salt thereof, when restarting treatment as per recommended dose modification guidelines.

In an embodiment of the present invention, the RCC patient is an advanced RCC patient. In a further embodiment, the advanced RCC patient is a first-line advanced RCC patient or a second-line RCC patient. In certain embodiments, the method of the present invention further comprises administering chemotherapy, radiotherapy, immunotherapy, or phototherapy, or any combinations thereof to a patient.

In one embodiment, the IO agent is an anti-PD-1 antibody. In some such embodiments, the anti-PD-1 antibody is nivolumab (MDX 1106), pembrolizumab (MK-3475), pidilizumab (CT-011), cemiplimab (REGN2810), tislelizumab (BGB-A317), spartalizumab (PDR001), sasanlimab (RN888), mAb15 (WO 2016/092419), MEDI-0680 (AMP-514), BGB-108, or AGEN-2034, JTX-4014, camrelizumab (SHR1210), sintilimab ((IBI308), toripalimab (JS001), dostarlimab (TSR-042, WBP-285), INCMGA00012 (MGA012), or AMP-224, or a combination thereof.

In some embodiments of the above methods, the IO agent is a PD-L1 antagonist. In certain embodiments, the PD-L1 antagonist inhibits the binding of PD-L1 to PD-1. In some embodiments, the PD-L1 binding antagonist inhibits the binding of PD-L1 to B7-1. In some embodiments, the PD-L1 binding antagonist inhibits the binding of PD-L1 to both PD-1 and B7-1. In a particular embodiment, the PD-L1 binding antagonist is an anti-PD-L1 antibody. In a more specific embodiment, the anti-PD-L1 antibody is avelumab (MSB0010718C), BMS-936559 (MDX-1105), AMP-714, atezolizumab (MPDL3280A), durvalumab (MED14736), envafolimab (KN035), cosibelimab (CK-301), CS-1001, SHR-1316, TQB2450 (CBT-502), BGB-A333 or an antibody comprising a VH region produced by the expression vector with ATCC Accession No. PTA-121183 and having the VL region produced by the expression vector with ATCC Accession No. PTA-121182, or a combination thereof.

In alternative embodiments of the above listed inventions, the axitinib dose is reduced rather than interrupted for a given number of days.

### BRIEF DESCRIPTION OF THE DRAWINGS

FIG. 1 depicts time to resolution (TTR) of any grade AE after temporary interruption or discontinuation by treatment cohort. The bar graph value represents the median. The designation n1 reflects the number of events that resolved and the designation n2 reflects the number of patients. The range value is the range of TTR for each AE within each cohort.

FIG. 2 depicts time to resolution of AEs designated at grade 3 or higher. The bar graph value represents the median. The designation n1 reflects the number of events that resolved and the designation n2 reflects the number of patients. The range value is the range of TTR for each AE within each cohort.

### DETAILED DESCRIPTION OF THE INVENTION

Each of the embodiments described below can be combined with any other embodiment described herein not inconsistent with the embodiment with which it is combined. Furthermore, each of the embodiments described herein envisions within its scope pharmaceutically acceptable salts of the small molecule compounds described herein. Accordingly, the phrase “or a pharmaceutically acceptable salt thereof” is implicit in the description of all small molecule compounds described herein.

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The term “about” when used to modify a numerically defined parameter means that the parameter may vary by as much as 10% above or below the stated numerical value for that parameter

As used herein, terms, including, but not limited to, “agent”, “component”, “composition”, “compound”, “substance”, “targeted agent”, “targeted therapeutic agent”, “therapeutic agent”, and “therapeutic antibody” may be used interchangeably to refer to the compounds used in the present invention, or combinations thereof.

An “immune-oncology agent” or “IO agent” means an oncology agent that works to kill tumor cells or cancer cells by harnessing the body's own immune system to kill the cells. IO agents work as checkpoint inhibitors.

“Biotherapeutic agent” means a biological molecule, such as an antibody or fusion protein, that blocks ligand/receptor signaling in any biological pathway that supports tumor maintenance and/or growth or suppresses the anti-tumor immune response.

A “chemotherapeutic agent” or “chemotherapy” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as.benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma and calicheamicin omegal (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-it inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDIS1NE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY 1 1 7018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, luteinizing hormone-releasing hormone (LHRFI) agonists such as leuprolide acetate (LUPRON® and ELIGARD®), goserelin acetate, buserelin acetate and tripterelin; anti-androgens such as fiutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RJVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

By “recommended dose modification guidelines” is meant such guidelines as they appear in the product label prescribing information. For example, the current US prescribing information for INLYTA (axitinib) currently provides (Section 2.2 Dose Modification Guidelines), that “[o]ver the course of treatment, management of some adverse drug reactions may require temporary interruption or permanent discontinuation and/or dose reduction of INLYTA therapy. If dose reduction from 5 mg twice daily is required, the recommended dose is 3 mg twice daily. If additional dose reduction is required, the recommended dose is 2 mg twice daily.”

Immune-Oncology (IO) Agents

One embodiment of the present invention comprises methods of managing an adverse event in an RCC patient being treated with axitinib in combination with an IO agent. IO agents work by harnessing the body's own immune system to kill tumor cells. They work as checkpoint inhibitors. Immune checkpoint proteins are found on the surface of T-cells, act as regulators of the immune system, and, in normal circumstances, prevent the immune system from attacking the body's own cells indiscriminately. However, tumors can leverage certain immune checkpoint pathways as a mechanism to evade an immune response. PD-1 is an example of an inhibitory checkpoint receptor protein found on the surface of T-cells that normally acts as an immune off-switch after interaction with the PD-1 ligand (PD-L1), a protein expressed on the surface of normal cells. However, PD-L1 is expressed by many types of tumor cells and is upregulated in some, thus activating the off-switch and protecting the malignant tumor cells from attack. IO agents, such as PD-1 antagonists and PD-L1 antagonists, that antagonize the interaction between PD-L1 on tumor cells with PD-1 on T cells overcomes this off-switch and allows the immune system to launch an anti-tumor response. IO agents can also target other receptors and ligands present on tumor cells (e.g., GITRL, 4-1BBL, CD70, CD155/CD112/CD113, MCH11, CD40, OX40L, PD-L2, and CD80/86) and on immune cells (e.g., GITR, 4-1BB, CD27, TIGIT, LAG3, TCR, CD40L, OX40, CTLA-4 and CD28).

In some embodiments, the 4-1BB agonist is utomilumab (PF-05082566), 1D8, 3EIor, 4B4, H4-1BB-M127, BBK2, 145501, antibody produced by cell line deposited as ATCC No. HB-11248, 5F4, C65-485, urelumab (BMS-663513), 20H4.9-IgG-1 (BMS-663031), 4E9, BMS-554271, BMS-469492, 3H3, BMS-469497, 3E1, 53A2, or 3B8. In some embodiments, OX40 agonist antibody is as described in, for example, U.S. Pat. No. 7,960,515, PCT Patent Application Publication Nos. WO2013028231 and WO2013/119202, and U.S. Patent Application Publication No. 20150190506.

PD-1 Axis Antagonists as IO Agents

As used herein, the term “PD-1 axis antagonist” refers to a molecule that interacts with and inhibits the interaction of a PD-1 axis binding partner (e.g., PD-1, PD-L1, PD-L2) with either one or more of its binding partners, for example so as to overcome or partially overcome T-cell dysfunction resulting from signaling on the PD-1 signaling axis—with a result being to restore, partially restore or enhance T-cell function (e.g., proliferation, cytokine production, target cell killing, survival). As used herein, a PD-1 axis antagonist includes a PD-1 antagonist, a PD-L1 antagonist, and/or a PD-L2 antagonist.

The term “PD-1 antagonist” as used herein refers to a molecule that interacts with PD-1 and decreases, blocks, inhibits, abrogates or interferes with signal transduction resulting from the interaction of PD-1 with one or more of its binding partners, such as PD-L1, PD-L2. In some embodiments, the PD-1 antagonist is a molecule that inhibits the binding of PD-1 to its binding partners. In a specific aspect, the PD-1 binding antagonist inhibits the binding of PD-1 to PD-L1 and/or PD-L2. For example, PD-1 antagonists include anti-PD-1 antibodies, antigen binding fragments thereof, immunoadhesins, fusion proteins, oligopeptides, small molecules, and other molecules that decrease, block, inhibit, abrogate or interfere with signal transduction resulting from the interaction of PD-1 with PD-L1 and/or PD-L2. In some embodiments, a PD-1 antagonist reduces the negative co-stimulatory signal mediated by or through cell surface proteins expressed on T lymphocytes mediated signaling through PD-1 so as to render a dysfunctional T-cell less non-dysfunctional. In some embodiments, the PD-1 antagonist is an anti-PD-1 antibody.

In some embodiments, the anti-PD-1 antibody useful for this invention is nivolumab (MDX 1106), pembrolizumab (MK-3475), pidilizumab (CT-011), cemiplimab (REGN2810), tislelizumab (BGB-A317), spartalizumab (PDR001), sasanlimab (RN888), mAb15 (WO 2016/092419), MEDI-0680 (AMP-514), BGB-108, or AGEN-2034, JTX-4014, camrelizumab (SHR1210), sintilimab ((IBI308), toripalimab (JS001), dostarlimab (TSR-042, WBP-285), INCMGA00012 (MGA012), or AMP-224, or a combination thereof.

Exemplary PD-1 antagonists include those described in U.S. 20130280265, U.S 20130237580, U.S. 20130230514, U.S. 20130109843, U.S. 20130108651, U.S. 20130017199, U.S. 20120251537, U.S. 20110271358, EP2170959B1, WO 2011/066342), WO 2015/035606, WO 2015/085847, WO 2015/112800, WO 2015/112900, WO 2016/092419, WO 2017/017623, WO 2017/024465,WO 2017/054646, WO 2017/071625, WO 2017/019846, WO 2017/132827, WO 2017/214092, WO 2018/013017, WO 2018/053106, WO 2018/055503, WO 2018/053709, WO 2018/068336, WO 2018/072743), the entire disclosures of which are incorporated herein by reference. Other exemplary PD-1 antagonists are described in Curran et al., PNAS 107: 4275, 2010; Topalian et al., New Engl. J. Med. 366: 2443, 2012; Brahmer et al., New Engl. J. Med. 366: 2455, 2012; Dolan et al., Cancer Control 21: 3, 2014; and Sunshine et al., Curr. Opin. in Pharmacol. 23: 32-38, 2015.

The term “PD-L1 antagonist” as used herein refers to a molecule that interacts with PD-L1 and decreases, blocks, inhibits, abrogates or interferes with signal transduction resulting from the interaction of PD-L1 with either one or more of its binding partners, such as PD-1, B7-1. In some embodiments, the PD-L1 antagonist inhibits the binding of PD-L1 to its binding partners. In a specific aspect, the PD-L1 antagonist inhibits the binding of PD-L1 to PD-1. In another specific aspect, the PD-L1 antagonist inhibits the binding of PD-L1 to PD-1 and/or B7-1. In another specific aspect, the PD-L1 antagonist inhibits the binding of PD-L1 to both PD-1 and B7-1.

In some embodiments, the PD-L1 antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, and/or B7-1. In some embodiments, a PD-L1 antagonist reduces the negative co-stimulatory signal mediated by or through cell surface proteins expressed on T lymphocytes mediated signaling through PD-L1 so as render a dysfunctional T-cell less non-dysfunctional. In some embodiments, a PD-L1 antagonist is an anti-PD-L1 antibody. In some embodiments, the PD-L1 antibody is a biosimilar, biobetter, or bioequivalent thereof.

In a particular embodiment, the PD-L1 binding antagonist is an anti-PD-L1 antibody. In a more specific embodiment, the anti-PD-L1 antibody is avelumab (MSB0010718C), BMS-936559 (MDX-1105), AMP-714, atezolizumab (MPDL3280A), durvalumab (MEDI4736), envafolimab (KN035), cosibelimab (CK-301), CS-1001, SHR-1316, TQB2450 (CBT-502), BGB-A333, YW243.55.570, or an antibody comprising a VH region produced by the expression vector with ATCC Accession No. PTA-121183 and having the VL region produced by the expression vector with ATCC Accession No. PTA-121182, or a combination thereof. Other exemplary PD-L1 binding antagonists are described in Sunshine et al., 2015, supra).

The term “PD-L2 antagonist” as used herein refers to a molecule that interacts with PD-L2 and decreases, blocks, inhibits, abrogates or interferes with signal transduction resulting from the interaction of PD-L2 with either one or more of its binding partners, such as PD-1. In some embodiments, a PD-L2 antagonist is a molecule that inhibits the binding of PD-L2 to its binding partners. In a specific aspect, the PD-L2 antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In some embodiments, a PD-L2 antagonist reduces the negative co-stimulatory signal mediated by or through cell surface proteins expressed on T lymphocytes mediated signaling through PD-L2 so as render a dysfunctional T-cell less non-dysfunctional. In some embodiments, a PD-L2 antagonist is a PD-L2 immunoadhesin.

A PD-1, PD-L1, and PD-L2 antagonist antibody useful in any of the treatment methods includes a monoclonal antibody (mAb), or antigen binding fragment thereof, which specifically binds to PD-1, PD-L1, or PD-L2. The mAb may be a human antibody, a humanized antibody or a chimeric antibody, and may include a human constant region. In some embodiments, the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)₂, scFv and Fv fragments.

In some embodiments the PD-1 axis antagonist (e.g., PD-1 antagonist, PD-L1 antagonist, or PD-L2 antagonist) is a small molecule antagonist. In some further embodiments the PD-1 axis binding antagonist (e.g., PD-L1 binding antagonist) is a chemical compound disclosed in PCT Publication No. WO 2015/033299 or WO 2015/033301 or a pharmaceutically acceptable salt thereof.

The term “antibody” as used herein refers to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses a polyclonal antibody, a monoclonal antibody, a chimeric antibody, a bispecific antibody, a dual-specific antibody, bifunctional antibody, a trispecific antibody, a multispecific antibody, a bispecific heterodimeric diabody, a bispecific heterodimeric IgG, a labeled antibody, a humanized antibody, a human antibody, and fragments thereof (such as Fab, Fab′, F(ab′)₂, Fv), single chain (ScFv) and domain antibodies (including, for example, shark and camelid antibodies), fusion proteins comprising an antibody, any other modified configuration of the immunoglobulin molecule that comprises an antigen recognition site, and antibody like binding peptidomimetics (ABiPs). An antibody includes an antibody of any class, such as IgG, IgA, or IgM (or sub-class thereof), but the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, a “bispecific antibody”, “dual-specific antibody”, “bifunctional antibody”, “heteromultimer”, “heteromultimeric complex”, “bispecific heterodimeric diabody” or a “heteromultimeric polypeptide” is a molecule comprising at least a first polypeptide and a second polypeptide, wherein the second polypeptide differs in amino acid sequence from the first polypeptide by at least one amino acid residue. In some instances, the bispecific is an artificial hybrid antibody having two different heavy chain region and light chain region. Preferably, the bispecific antibody has binding specificity for at least two different ligands, antigens or binding sites. Accordingly, the bispecific antibodies can bind simultaneously two different antigens. The two antigen binding sites of a bispecific antibody bind to two different epitopes, which may reside on the same or different protein targets, e.g., tumor target.

The term “immunoglobulin” (Ig) is used interchangeably with “antibody” herein.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

An “amino-acid modification” at a specified position, e.g., of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The phrase “substantially reduced,” “substantially different,” or “substantially inhibit” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10 percent of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

A “fusion protein” and a “fusion polypeptide” refer to a polypeptide having two portions covalently linked together, where each of the portions is a polypeptide having a different property. The property may be a biological property, such as activity in vitro or in vivo. The property may also be simple chemical or physical property, such as binding to a target molecule, catalysis of a reaction, etc. The two portions may be linked directly by a single peptide bond or through a peptide linker but are in reading frame with each other.

A “PD-1 oligopeptide,” “PD-L1 oligopeptide,” or “PD-L2 oligopeptide” is an oligopeptide that binds, preferably specifically, to a PD-1, PD-L1 or PD-L2 negative costimulatory polypeptide, respectively, including a receptor, ligand or signaling component, respectively, as described herein. Such oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more. Such oligopeptides may be identified using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002, 1984; Geysen et al., Proc. Natl. Acad. Sci. U.S.A., 82:178-182, 1985; Geysen et al., in Synthetic Peptides as Antigens, 130-149, 1986; Geysen et al., J. Immunol. Metk, 102:259-274, 1987; Schoofs et al., J. Immunol, 140:611-616, 1988, Cwirla, S. E. et al., Proc. Natl. Acad. Sci. USA, 87:6378, 1990; Lowman, H. B. et al., Biochemistry, 30:10832, 1991; Clackson, T. et al., Nature, 352: 624, 1991; Marks, J. D. et al., J. Mol. Biol, 222:581, 1991; Kang, A. S. et al., Proc. Natl. Acad. Sci. USA, 88:8363, 1991), and Smith, G. P., Current Opin. Biotechnol, 2:668, 1991.

As used herein, “metastasis” or “metastatic” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “cancer”, “cancerous”, or “malignant” refers to or describes the physiological condition in a patient that is typically characterized by unregulated cell growth. As used herein “cancer” refers to any malignant and/or invasive growth or tumor caused by abnormal cell growth. As used herein “cancer” refers to solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one. Examples of cancer include, but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, follicular lymphoma (FL), diffuse large B-cell lymphoma (DLCBCL), acute myeloid leukemia (AML), multiple myeloma, gastrointestinal (tract) cancer, renal cancer, ovarian cancer, liver cancer, kidney cancer, prostate cancer, castration resistant prostate cancer (CRPC), thyroid cancer, melanoma, chondrosarcoma, neuroblastoma, pancreatic cancer, glioblastoma, multiformer, cervical cancer, brain cancer, stomach cancer, bladder cancer, hepatoma, breast cancer, colon carcinoma, and head and neck cancer.

As used herein, “in combination with” or “in conjunction with” refers to administration of one treatment modality in addition to at least one other treatment modality. As such, “in combination with” or “in conjunction with” refers to administration of one treatment modality before, during, or after administration of at least one other treatment modality to the individual.

A “patient” to be treated according to this invention includes any warm-blooded animal, such as, but not limited to human, monkey or other lower-order primate, horse, dog, rabbit, guinea pig, or mouse. For example, the patient is human.

The term “advanced”, as used herein, includes locally advanced (non-metastatic) disease and metastatic disease.

The term “pharmaceutical composition” refers to a preparation which is in such form as to permit the biological activity of the active ingredient to be effective, and which contains no additional components which are unacceptably toxic to a patient to which the formulation would be administered. Such formulations are sterile. “Pharmaceutically acceptable” excipients (vehicles, additives) are those which can reasonably be administered to a patient to provide an effective dose of the active ingredient employed.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

The term “treat” or “treating” a cancer as used herein means to administer a therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the purposes of this invention, beneficial or desired clinical results include, but are not limited to, one or more of the following: reducing the proliferation of (or destroying) neoplastic or cancerous cells; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (200)).

The terms “treatment regimen”, “dosing protocol” and “dosing regimen” are used interchangeably to refer to the dose and timing of administration of each therapeutic agent in a combination of the invention.

As used herein, an “effective dosage” or “effective amount” of drug, compound or pharmaceutical composition is an amount sufficient to affect any one or more beneficial or desired, including biochemical, histological and/or behavioral symptoms, of the disease, its complications and intermediate pathological phenotypes presenting during development of the disease. For therapeutic use, a “therapeutically effective amount” refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) relieving to some extent (or, preferably, eliminating) one or more signs or symptoms associated with the cancer, (5) decreasing the dose of other medications required to treat the disease, and/or (6) enhancing the effect of another medication, and/or delaying the progression of the disease of patients. An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another drug, compound or pharmaceutical composition.

“Tumor” as it applies to a patient diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukemia's (cancers of the blood) generally do not form solid tumors (National Cancer Institute, Dictionary of Cancer Terms).

A “nonstandard clinical dosing regimen” as used herein, refers to a regimen for administering a substance, agent, compound or composition, which is different to the amount, dose or schedule typically used for that substance, agent, compound or composition in a clinical setting. A “non-standard clinical dosing regimen”, includes a “non-standard clinical dose” or a “nonstandard dosing schedule”.

A “low dose amount regimen” as used herein refers to a dosing regimen where one or more of the substances, agents, compounds or compositions in the regimen are dosed at a lower amount or dose than typically used in a clinical setting for that agent, for example when that agent is dosed as a singleton therapy.

The term “pharmaceutically acceptable salt” as used herein, refers to pharmaceutically acceptable organic or inorganic salts of a compound of the invention. Some embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, trifluoroacetate and xinofoate salts.

“Carriers” as used herein include pharmaceutically acceptable carriers, excipients, or stabilizers that are nontoxic to the cell or patient being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Exemplary methods and materials are described herein, although methods and materials similar or equivalent to those described herein can also be used in the practice or testing of the invention. The materials, methods, and examples are illustrative only and not intended to be limiting.

In accordance with the present invention, an amount of a first compound or component is combined with an amount of a second compound or component, and the amounts together are effective in the treatment of cancer. The amounts, which together are effective, will relieve to some extent one or more of the symptoms of the disorder being treated. In reference to the treatment of cancer, an effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression”.

Dosage Forms and Regimens

Administration of the compounds of the invention may be affected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated and may include single or multiple doses. It is to be further understood that for any particular patient, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the therapeutic agent are well-known in the relevant art and would be understood to be encompassed by the skilled artisan once provided the teachings disclosed herein.

The amount of the compound of the invention administered will be dependent on the patient being treated, the severity of the disorder or condition, the rate of administration, the disposition of the compound and the discretion of the prescribing physician.

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of breast cancer, requiring treatment, and the presence of other medications.

The practice of the method of this invention may be accomplished through various administration or dosing regimens. The compounds of the combination of the present invention can be administered intermittently, concurrently or sequentially. In an embodiment, the compounds of the combination of the present invention can be administered in a concurrent dosing regimen.

The specification is sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The invention will be more fully understood by reference to the following examples. It should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

Materials and Methods

Overview

This was a post-hoc analysis of data from 5 studies: two studies were randomized trials of axitinib monotherapy with sorafenib as the comparator (studies A4061032 [AXIS; NCT00678392] in the second-line setting, and A4061051 [NCT00920816] in the first-line setting) (Rini et al., Lancet 378: 1931-1939, 2011; Hutson et al., Lancet Oncology 14: 1287-1294, 2013); two studies were of axitinib in combination with avelumab (studies B9991002 [JAVELIN Renal 100; NCT02493751] and 69991003 [JAVELIN Renal 101; NCT02684006], both in the first-line setting, 69991002 was a single-arm study, whereas B9991003 was a randomized trial with sunitinib as the comparator) (Choueiri et al., Lancet Oncol 19: 451-460, 2018; Motzer et al., N Engl J Med 380: 1103-1115, 2019); and one study was a single-arm trial of axitinib in combination with pembrolizumab (study A4061079 [NCT02853331] in the first-line setting) (Atkins et al., Lancet Oncol 19: 405-415, 2018). Studies A4061051, 69991002, and 69991003 are ongoing; therefore, data cutoffs of 30 Oct. 2018, 3 Apr. 2018, and 20 Jun. 2018, respectively, were used for this analysis. Data from 24 patients in China who participated in study A4061051 were excluded due to regulatory requirements.

Patient Eligibility and Dosing

Detailed descriptions of the methodology and results of each of the studies have been reported previously (Rini et al., Lancet 2011, supra; Hutson et al., Lancet Oncol 2013, supra; Choueiri et al. Lancet Oncol 2018, supra; Motzer et al., N Engl J Med 2019, supra; Atkins et al., Lancet Oncol, 2018, supra). Briefly, across all studies, male and female patients were included if they were aged years (20 years in Japan), had histologically confirmed aRCC with a component of clear cell type and evidence of measurable disease, received no prior systemic first-line therapy or there was weeks since the end of prior systemic therapy, an Eastern Cooperative Oncology Group performance status of 0 or 1, and adequate organ function. Across all studies, the starting dose of axitinib was 5 mg twice-daily (BID) with dose escalation up to a maximum of 10 mg BID based on patient tolerability. Avelumab was administered at 10 mg/kg of bodyweight every 2 weeks, with no dose modification permitted. Pembrolizumab was administered at 2 mg/kg of bodyweight every 3 weeks, with no dose modification permitted. The starting dose for sorafenib was 400 mg BID and dose escalation was not permitted. The starting dose for sunitinib was 50 mg once-daily on a schedule of 4 weeks on treatment and 2 weeks off treatment, with no dose escalation permitted. Each of the original studies was conducted in accordance with the International Council for Harmonisation Good Clinical Practice Guidelines and the Declaration of Helsinki. All patients provided written informed consent. Patient consent forms, the protocol, and any protocol amendments were reviewed and approved by the institutional review board or independent ethics committee of each participating center.

Data Analysis

Five of the most common AEs associated with axitinib treatment were assessed: diarrhea; fatigue; hypertension; nausea; and palmar-plantar erythrodysesthesia syndrome (PPE). Incidences of treatment-emergent AEs were taken from the full published articles for each study. AEs were classified and graded per the Common Terminology Criteria for Adverse Events version 3.0 or the National Cancer Institute Common Terminology Criteria for Adverse Events version 4.03, as used in each individual study. Incidences of AEs were reported for any grade and for grade ≥3.

To determine the TTR for each of the AEs, data were pooled for different treatment cohorts as follows: patients who received axitinib monotherapy (axitinib monotherapy cohort); patients who received axitinib in combination with IO therapy (axitinib+10 cohort); and patients in comparator treatment arms who received another TKI as monotherapy, i.e., sorafenib or sunitinib (other TKI cohort). TTR was calculated for all causality, treatment-emergent AEs. For each AE reported for patients in each pooled cohort, the action taken on presentation of the AE was documented. TTR was calculated as the time from temporary treatment interruption or treatment discontinuation to resolution of the AE, with outcome status confirmed as resolved. TTR was determined for AEs of any grade and for grade Data were descriptive only; no statistical comparisons were made.

Results

The published incidences of each of the treatment-emergent AEs for any grade and for grade by study and by treatment arm, are summarized in Table 2 and Table 3.

TABLE 2
Incidences of treatment-emergent adverse
events at any grade and grade ≥3 for each
monotherapy study and treatment arm

| | Monotherapy Study and Treatment Arms | | | |
|---|---|---|---|---|
| | 1032 | | 1051 | |
| | (Rini et al., | | (Hutson et al., | |
| | 2011, supra) | | 2017, supra) | |
| Adverse | Axitinib | Sorafenib | Axitinib | Sorafenib |
| event, n (%) | n = 359 | n = 355 | n = 189 | n = 96 |

Diarrhea

| | | | | | | | | |
|---|---|---|---|---|---|---|---|---|
| Any grade | 197 | (54.9) | 189 | (53.2) | 100 | (52.9) | 40 | (41.7) |
| Grade ≥3 | 38 | (10.6) | 26 | (7.3) | 19 | (10.1) | 5 | (5.2) |

Fatigue

| | | | | | | | | |
|---|---|---|---|---|---|---|---|---|
| Any grade | 140 | (39.0) | 112 | (31.5) | 65 | (34.4) | 27 | (28.1) |
| Grade ≥3 | 41 | (11.4) | 18 | (5.1) | 12 | (6.3) | 1 | (1.0) |

Hypertension

| | | | | | | | | |
|---|---|---|---|---|---|---|---|---|
| Any grade | 145 | (40.4) | 103 | (29.0) | 93 | (49.2) | 30 | (31.3) |
| Grade ≥3 | 56 | (15.6) | 39 | (11.0) | 25 | (13.2) | 1 | (1.0) |

Nausea

| | | | | | | | | |
|---|---|---|---|---|---|---|---|---|
| Any grade | 116 | (32.3) | 77 | (21.7) | 38 | (20.1) | 16 | (16.7) |
| Grade ≥3 | 9 | (2.5) | 4 | (1.1) | 2 | (1.1) | 2 | (2.1) |

PPE

| | | | | | | | | |
|---|---|---|---|---|---|---|---|---|
| Any grade | 98 | (27.3) | 181 | (51.0) | 51 | (27.0) | 37 | (38.5) |
| Grade ≥3 | 18 | (5.0) | 57 | (16.1) | 14 | (7.4) | 15 | (15.6) |

TABLE 3
Incidences of treatment-emergent adverse
events at any grade and grade ≥3 for each
combination therapy study and treatment arm

| | Combination Study and Treatment Arms | | | |
|---|---|---|---|---|
| | 1002 | | | |
| | (Choueiri et | 1003 | | 1079 |
| | al., 2018, | (Motzer et al., | | (Atkins et al., |
| | supra) | 2019, supra) | | 2018, supra |
| | Axitinib | Axitinib | | Axitinib |
| Adverse | avelumab | avelumab | Sunitinib | pembrolizumab |
| event, n (%) | n = 55 | n = 434 | n = 439 | n = 52 |

Diarrhea

| | | | | | | | | |
|---|---|---|---|---|---|---|---|---|
| Any grade | 35 | (63.6) | 270 | (62.2) | 209 | (47.6) | 44 | (84.6) |
| Grade ≥3 | 2 | (3.6) | 29 | (6.7) | 12 | (2.7) | 5 | (9.6) |

Fatigue

| | | | | | | | | |
|---|---|---|---|---|---|---|---|---|
| Any grade | 28 | (50.9) | 180 | (41.5) | 176 | (40.1) | 41 | (78.8) |
| Grade ≥3 | 2 | (3.6) | 15 | (3.5) | 16 | (3.6) | 5 | (9.6) |

Hypertension

| | | | | | | | | |
|---|---|---|---|---|---|---|---|---|
| Any grade | 26 | (47.3) | 215 | (49.5) | 158 | (36.0) | 28 | (53.8) |
| Grade ≥3 | 16 | (29.1) | 111 | (25.6) | 75 | (17.1) | 12 | (23.1) |

Nausea

| | | | | | | | | |
|---|---|---|---|---|---|---|---|---|
| Any grade | 15 | (27.3) | 148 | (34.1) | 172 | (39.2) | 22 | (42.3) |
| Grade ≥3 | 1 | (1.8) | 6 | (1.4) | 7 | (1.6) | 1 | (1.9) |

PPE

| | | | | | | | | |
|---|---|---|---|---|---|---|---|---|
| Any grade | 17 | (30.9) | 145 | (33.4) | 148 | (33.7) | 19 | (36.5) |
| Grade ≥3 | 4 | (7.3) | 25 | (5.8) | 19 | (4.3) | 2 | (3.8) |

When data were pooled by treatment, the axitinib monotherapy cohort comprised 532 patients, the axitinib+10 cohort 541 patients, and the other TKI cohort (sorafenib and sunitinib) 882 patients. The action taken for each occurrence of the AE of interest by treatment cohort is shown in Table 4. Overall, ‘no action’ was the most common action taken for each AE in each treatment cohort, and ‘stopped temporarily’ was the second most common action taken. Across all AEs and treatment cohorts, an action of ‘permanently discontinued’ occurred in <1.0% of cases. Action taken was determined as ‘not applicable’ when the associated drug was already permanently discontinued when the AE occurred. N=the number of patients in each pooled cohort.

TABLE 4
Action taken for each occurrence of each AE by treatment cohort

| | | Axitinib | | | | | |
|---|---|---|---|---|---|---|---|
| | | monotherapy | | Axitinib + IO | | Other TKI | |
| Adverse | | n = 532 | | n = 541 | | n = 882 | |
| event | Action taken | n (%) | | n (%) | | n (%) | |
| Diarrhea | | n = 1249 | | n = 974 | | n = 1050 | |
| | No action taken | 909 | (72.8) | 795 | (81.6) | 911 | (86.8) |
| | Dose reduced | 66 | (5.3) | 14 | (1.4) | 29 | (2.8) |
| | Stopped temporarily | 273 | (21.9) | 149 | (15.3) | 86 | (8.2) |
| | Permanently | 1 | (0.1) | 3 | (0.3) | 4 | (0.4) |
| | discontinued | | | | | | |
| | Stopped temporarily | 0 | | 4 | (0.4) | 0 | |
| | and reduced | | | | | | |
| | Not applicable | 0 | | 9 | (0.9) | 2 | (0.2) |
| | Unknown | 0 | | 0 | | 17 | (1.6) |
| | No action taken | 909 | (72.8) | 795 | (81.6) | 911 | (86.8) |
| Fatigue | | n = 490 | | n = 479 | | n = 530 | |
| | No action taken | 409 | (83.5) | 399 | (83.3) | 434 | (81.9) |
| | Dose reduced | 27 | (5.5) | 11 | (2.3) | 15 | (2.8) |
| | Stopped temporarily | 49 | (10.0) | 46 | (9.6) | 57 | (10.8) |
| | Permanently | 4 | (0.8) | 4 | (0.8) | 4 | (0.8) |
| | discontinued | | | | | | |
| | Stopped temporarily | 0 | | 3 | (0.6) | 0 | |
| | and reduced | | | | | | |
| | Not applicable | 0 | | 16 | (3.3) | 2 | (0.4) |
| | Unknown | 0 | | 0 | | 18 | (3.4) |
| | No action taken | 409 | (83.5) | 399 | (83.3) | 434 | (81.9) |
| | Dose reduced | 27 | (5.5) | 11 | (2.3) | 15 | (2.8) |
| Hypertension | | n = 561 | | n = 503 | | n = 470 | |
| | No action taken | 307 | (54.7) | 378 | (75.1) | 353 | (75.1) |
| | Dose reduced | 27 | (4.8) | 17 | (3.4) | 16 | (3.4) |
| | Stopped temporarily | 226 | (40.3) | 102 | (20.3) | 77 | (16.4) |
| | Permanently | 1 | (0.2) | 3 | (0.6) | 1 | (0.2) |
| | discontinued | | | | | | |
| | Stopped temporarily | 0 | | 2 | (0.4) | 0 | |
| | and reduced | | | | | | |
| | Not applicable | 0 | | 1 | (0.2) | 2 | (0.4) |
| | Unknown | 0 | | 0 | | 21 | (4.5) |
| Nausea | | n = 352 | | n = 284 | | n = 424 | |
| | No action taken | 312 | (88.6) | 241 | (84.9) | 368 | (86.8) |
| | Dose reduced | 8 | (2.3) | 5 | (1.8) | 10 | (2.4) |
| | Stopped temporarily | 32 | (9.1) | 29 | (10.2) | 30 | (7.1) |
| | Permanently | 0 | | 0 | | 3 | (0.7) |
| | discontinued | | | | | | |
| | Stopped temporarily | 0 | | 0 | | 0 | |
| | and reduced | | | | | | |
| | Not applicable | 0 | | 9 | (3.2) | 2 | (0.5) |
| | Unknown | 0 | | 0 | | 11 | (2.6) |
| PPE | | n = 545 | | n = 517 | | n = 915 | |
| | No action taken | 316 | (58.0) | 318 | (61.5) | 612 | (66.9) |
| | Dose reduced | 17 | (3.1) | 31 | (6.0) | 71 | (7.8) |
| | Stopped temporarily | 211 | (38.0) | 154 | (29.8) | 206 | (22.5) |
| | Permanently | 1 | (0.2) | 3 | (0.6) | 8 | (0.9) |
| | discontinued | | | | | | |
| | Stopped temporarily | 0 | | 9 | (1.7) | 0 | |
| | and reduced | | | | | | |

| | | | | | |
|---|---|---|---|---|---|
| Not applicable | 0 | 2 | (0.4) | 4 | (0.4) |

| | | | | |
|---|---|---|---|---|
| Unknown | 0 | 0 | 12 | (1.3) |

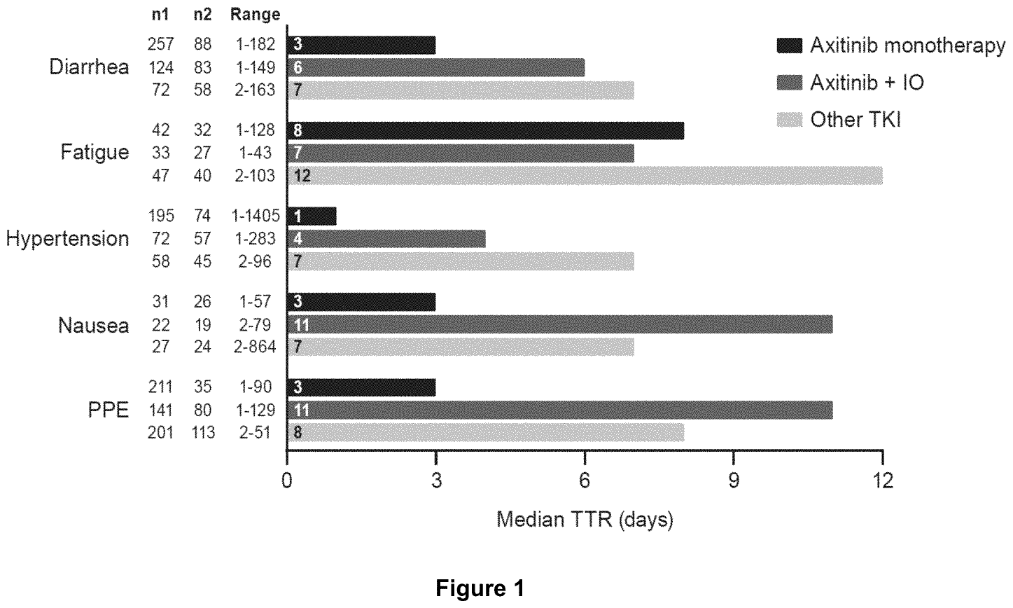

The median TTRs following treatment interruption or discontinuation for each AE of any grade by treatment cohort are shown in FIG. 1. In the axitinib monotherapy cohort, median (interquartile range [IQR]) TTR was 3 (1-6) days for diarrhea, 8 (4-16) days for fatigue, 1 (1-3) days for hypertension, 3 (1-7) days for nausea, and 3 (3-5) days for PPE. In the axitinib+10 cohort, median (IQR) TTR was 4-11 days for all of the AEs (diarrhea, 6 [3-12] days; fatigue, 7 [3-10] days; hypertension, 4 [2-14] days; nausea, 11 [4-16] days; PPE, 11 [6-19] days). With the exception of fatigue, median TTR for each AE in the axitinib monotherapy cohort was shorter than in the axitinib+10 cohort. In the other TKI cohort, median (IQR) TTR was 7-12 days for all of the AEs (diarrhea, 7 [3-15] days; fatigue, 12 [6-24] days; hypertension, 7 [3-14] days; nausea, 7 [4-14] days; PPE, 8 [5-15] days). No AE had a longer median TTR in the axitinib monotherapy cohort versus the other TKI cohort.

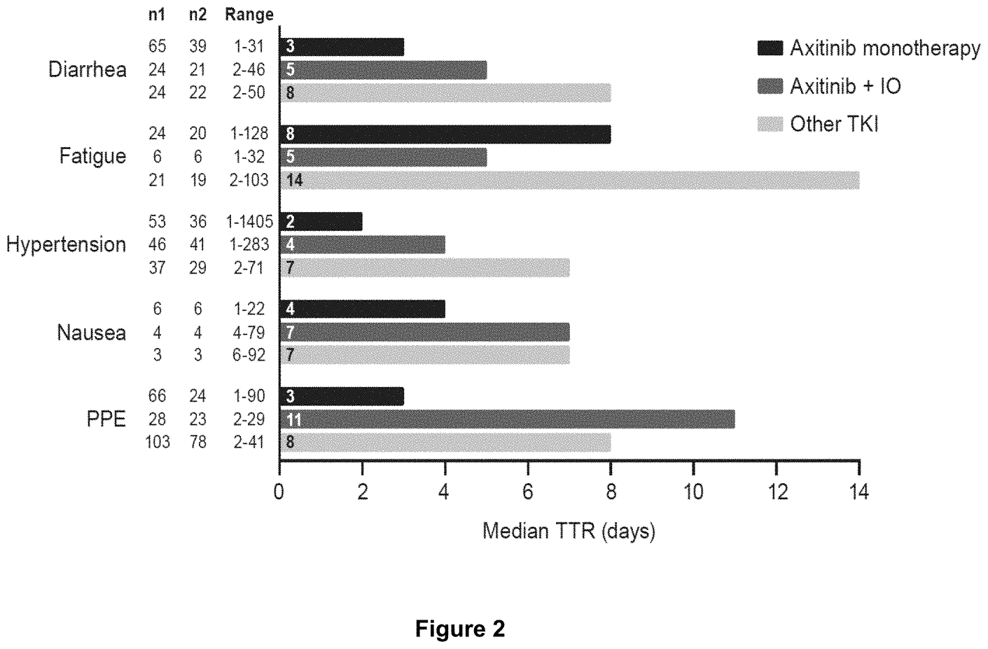

This pattern was repeated when only AEs of grade were considered (FIG. 2). Median TTRs for each AE in each treatment cohort were similar to the TTRs at any grade. In the axitinib monotherapy cohort, median (IQR) TTR was 3 (2-6) days for diarrhea, 8 (5-16) days for fatigue, 2 (1-8) days for hypertension, 4 (2-15) days for nausea, and 3 (1-6) days for PPE. In the axitinib+10 cohort, median (IQR) TTR was 4-11 days for all AEs (diarrhea, 5 [4-9] days; fatigue, 5 [3-10] days; hypertension, 4 [2-10] days; nausea, 7 [5-44] days; PPE, 11 [8-15] days). With the exception of fatigue, median TTR in the axitinib monotherapy group was shorter than in the axitinib+10 group. In the other TKI cohort, median TTR was 7-14 days for all AEs (diarrhea, 8 [4-10] days; fatigue, 14 [7-24] days; hypertension, 7 [3-14] days; nausea, 7 [6-92] days; PPE, 8 [5-14] days). No AE had a longer median TTR in the axitinib monotherapy cohort versus the other TKI cohort.

Discussion

The TTR of TKI toxicity has several implications for clinical practice. A shorter toxicity resolution reduces the burden of cumulative toxicity on a patient. Further, in combination regimens where both components may cause a given toxicity, the TTR may assist in determining toxicity etiology. Data from the current analysis demonstrated that treatment-emergent AEs commonly related to axitinib generally resolved quicker for axitinib monotherapy following treatment interruption than for other TKI monotherapies, and also for axitinib monotherapy compared with combined axitinib-IO therapy. Importantly, given that the longer TTR of other single agent TKIs would likely to extend to the combination of those TKIs with IO agents, axitinib-based IO combinations may allow for easier determination of toxicity etiology and guide management strategies.

The majority of TTRs associated with combined axitinib and IO therapy were longer than for axitinib monotherapy, as might be expected when comparing a combination therapy with a monotherapy. However, the TTR for fatigue was longer with axitinib monotherapy compared with combined axitinib and IO therapy, irrespective of AE severity. There are several possible reasons for this. Firstly, the number of events for some of the AEs were low. Secondly, the resolution of fatigue could have been confounded by other toxicities, disease-related issues such as disease progression, or concomitant medications. Thirdly, cancer-related fatigue is a multifactorial event (Weis, Expert Rev Pharmacoecon Outcomes Res 11: 441-446, 2011; O'Higgins et al., Support Care Cancer 26: 3353-3364, 2018; Yang et al., Cells 8: 738, 2019). The resolution of fatigue may therefore not be solely dependent on stopping treatment, but may also involve other factors such as patient lifestyle or mental health status (Dolgoy et al. Eur J Cancer Care (Engl) 28: e13048, 2019; Zou et al., J Clin Nurs 27: e1412-e8, 2018; Scott and Posmontier, Holist Nurs Pract 31: 66-79, 2017).

The shorter TTR for all the AEs when axitinib monotherapy was compared with other TKI therapies is most likely due to the shorter half-life of axitinib versus sorafenib and sunitinib. After a single 5 mg dose, the half-life of axitinib is 2.5-6.1 hr, compared with 25-48 hr for sorafenib and 40-60 hr for sunitinib. The shorter half-life of axitinib versus sorafenib and sunitinib, and other TKIs such as cabozantinib (˜99 hr), and lenvatinib (˜28 hr), may provide an advantage when considering the AE management of combined TKI and IO therapy. Overlapping toxicities represent a problem for combined TKI and IO therapy, particularly when trying to determine if an AE is TKI-related or immune-related. Temporarily stopping TKI treatment and observing whether the AE improves or resolves is a potential first step in determining the etiology. Compared with a TKI with a longer half-life, a TKI with a short half-life such as axitinib would enable faster resolution of TKI-related AEs, thereby potentially permitting earlier identification of the etiology of the AE and earlier implementation of an appropriate management strategy. The length of time an AE is given to resolve could be based on recommendations and guidance as well as clinical judgment, and additional investigations may be required before the AE can be confirmed as immune-related.

The data were descriptive only and no statistical comparisons were made among treatment cohorts. The ease of identifying AE etiology with combined axitinib and 10 therapy, i.e., axitinib- or immune-related, may be of most benefit for axitinib-related toxicities where the addition of 10 to axitinib worsens the AE or increases its frequency. Such examples may include hypothyroidism, mucositis, rash, or laboratory abnormalities, in particular hepatic toxicities.

Conclusion

In summary, data from 5 randomized or single arm INLYTA monotherapy or combination studies were analyzed to assess the time from INLYTA interruption to resolution (TTR) of all grade and Grade diarrhea, fatigue, hypertension, nausea, and palmar-plantar erythrodysesthesia syndrome (PPE). The INLYTA monotherapy cohort comprised 532 patients and the INLYTA combination cohort with avelumab or pembrolizumab comprised 541 patients. Median TTR for all grade adverse reactions in the INLYTA monotherapy cohort ranged from 1-3 days, except for fatigue (8 days). For all grade diarrhea, hypertension, nausea, fatigue, and PPE, median TTRs in the INLYTA combination cohort ranged from 4-11 days. Median TTR for Grade adverse reactions in the INLYTA monotherapy cohort ranged from 2-4 days, except for fatigue (8 days). For Grade diarrhea, hypertension, nausea, fatigue, and PPE, median TTRs in the INLYTA combination cohort ranged from 4-11 days.

Axitinib monotherapy-related toxicities resolved more quickly upon treatment interruption than for other single agent TKIs, or axitinib and 10 combination therapy. The early identification of the underlying etiology of a given AE with combined axitinib and 10 treatment, i.e., axitinib- or immune-related, may enable the earlier implementation of an appropriate AE management strategy.

### Example 2: Adverse Event Management Among Advanced Renal Cell Carcinoma Patients Receiving First-Line Axitinib in Combination with Avelumab or Pembrolizumab

Overview

A study was conducted to assess how dose reductions or treatment interruptions related to axitinib were implemented to manage and resolve adverse events occurring among patients with aRCC treated with first-line axitinib in combination with avelumab or pembrolizumab. The specific objectives of the study were as follows: Describe AEs experienced among patients with advanced RCC who received first-line axitinib in combination with IO therapies. This information included: type and seriousness of AEs (ie, diarrhea, fatigue, hypertension, nausea, palmar plantar erythrodysesthesia [PPE; hand-foot syndrome]); proportion of patients who experienced repeated AEs; and time from treatment initiation to AE onset, overall and by type and seriousness of AEs. Among patients with advanced RCC who developed incident AEs while receiving first-line axitinib in combination with IO therapies, the management strategies were characterized and were stratified by type and seriousness of AEs. Data was characterized with respect to the proportion of patients who used each of the following management strategies: no action for axitinib and IO therapy; no action for axitinib, but treatment modification for IO therapy (ie, treatment interruption, treatment discontinuation); axitinib dose reduction, but no action for IO therapy; axitinib treatment interruption, but no action for IO.

Materials and Methods

A retrospective chart review included adult aRCC patients treated with first-line axitinib+IO (avelumab or pembrolizumab) and had documented frequently reported axitinib-related AEs of fatigue, diarrhea, nausea, hypertension, or palmar-plantar erythrodysesthesia (PPE). Patient characteristics, AEs experienced, and assessment of strategies to manage AEs (no action vs axitinib modifications including dose reduction and/or treatment interruption) were described.

Study Endpoints
Type of AEs (i.e., fatigue, diarrhea, nausea, hypertension, or PPE)
AE characteristics (e.g., severity and seriousness of AE)
Type of AE management strategies
AE resolution/improvement (as reported final disposition of AE), described between focal AE management strategies (axitinib modifications [dose reduction and/or treatment interruption] vs. no action)
Time to AE resolution/improvement, described in two ways:
   Time from date of AE onset to date of documented AE resolution/improvement
   Time from date of management strategy initiation (for axitinib modifications) to date of AR resolution/improvement

Statistical Analysis

Baseline patient demographic and clinical characteristics (prior to treatment with axitinib+IO therapy), AE characteristics, AE management strategies used, AE resolution/improvement, and time to AE resolution/improvement were described and summarized. Continuous variables were summarized with mean (+/−standard deviation [SD]) and median (interquartile range [IQR]) values, which categorical variables were summarized with frequency distributions.

AE resolution/improvement among AEs treated with axitinib modifications vs. no action were described and compared across overall and among severe AEs via Chi-squared test or Fisher's exact test (if any category has <5 patients).

Results

Among 481 patient charts (median age 63 years, 67% male, 74% White) abstracted by 201 oncologists (67% community-based, 37% academic-based), 131 and 350 patients received axitinib+avelumab and axitinib+pembrolizumab, respectively; 83% patients remained on first-line at time of chart abstraction. Of the 209 (44%) patients with documented International Metastatic RCC Database Consortium (IMDC) risk scores, 11%, 52%, and 37% had favorable, intermediate, and poor risk, respectively. Baseline patient characteristics among patients with aRCC who experienced AEs while treated with axitinib+IO therapy are shown in Table 5.

TABLE 5
Baseline Patient Characteristics among Patients with aRCC who
Experiences AEs while Treated with Axitinib + IO Therapy¹

| | First-Line Therapy | |
|---|---|---|
| | Axitinib + | Axitinib + |
| Overall | Avelumab | Pembro |
| N - 481 | N - 131 | N - 350 |

Age at index² (years)

| Mean ± SD | 61.9 ± 9.5 | | 59.4 ± 10.3 | | 62.9 ± 9.1 | |
|---|---|---|---|---|---|---|
| Median (IQR) | 62.6 | (56.3, 69.3) | 60.1 | (52.3, 66.6) | 63.4 | (58.2, 68.6) |
| Male, n (%) | 320 | (66.5) | 82 | (62.6) | 238 | (68.0) |

Race/Ethnicity,³ n (%)

| | | | | | | |
|---|---|---|---|---|---|---|
| White | 358 | (74.4) | 102 | (77.9) | 256 | (73.1) |
| Black/African-American | 76 | (15.8) | 18 | (13.7) | 58 | (16.6) |
| Hispanic/Latino | 24 | (5.0) | 3 | (2.3) | 21 | (6.0) |
| Other⁴ | 21 | (4.4) | 9 | (6.9) | 12 | (3.4) |
| Unknown | 6 | (1.2) | 0 | (0.0) | 6 | (1.7) |

Time from aRCC diagnosis to index (months)

| Mean + SD | 1.8 ± 6.2 | | 1.3 ± 2.8 | | 2.0 ± 7.1 | |
|---|---|---|---|---|---|---|
| Median (IQR) | 0.5 | (0.2, 1.0) | 0.5 | (0.1, 1.1) | 0.5 | (0.3, 1.0) |

Nephrectomy prior to index, n (%)

| | | | | | | |
|---|---|---|---|---|---|---|
| Yes | 154 | (32.0) | 39 | (29.8) | 115 | (32.9) |
| No | 290 | (60.3) | 74 | (56.5) | 216 | (61.7) |
| Unknown | 37 | (7.7) | 18 | (13.7) | 19 | (5.4) |

Number of metastases, n (%)

| | | | | | | |
|---|---|---|---|---|---|---|
| 0 | 36 | (7.5) | 16 | (12.2) | 20 | (5.7) |
| 1 | 126 | (26.2) | 31 | (23.7) | 95 | (27.1) |
| >1 | 319 | (66.3) | 84 | (64.1) | 235 | (67.1) |

Largest primary tumor dimension (cm)

| Mean ± SD | 4.0 ± 2.9 | | 3.6 ± 2.1 | | 4.2 ± 3.1 | |
|---|---|---|---|---|---|---|
| Median (IQR) | 4.0 | (2.0, 5.0) | 3.0 | (2.0, 4.0) | 4.0 | (3.0, 5.0) |
| Unknown | 94 | (19.5) | 27 | (20.6) | 67 | (19.1) |

Sarcomatoid differentiation, n (%)

| | | | | | | |
|---|---|---|---|---|---|---|
| Yes | 60 | (12.5) | 29 | (22.1) | 31 | (8.9) |
| No | 372 | (77.3) | 81 | (61.8) | 291 | (83.1) |
| Unknown | 49 | (10.2) | 21 | (16.0) | 28 | (8.0) |
| IMDC risk score,⁵ n (%) | 209 | (43.5) | 40 | (30.5) | 169 | (48.3) |
| Favorable | 22 | (10.5) | 5 | (12.5) | 17 | (10.1) |
| Intermediate | 109 | (52.2) | 21 | (52.5) | 88 | (52.1) |
| Poor | 78 | (37.3) | 14 | (35.0) | 64 | (37.9) |

¹Patient characteristics were assessed as of the index date or on the date closest to the index during the baseline period, which was defined as the 12-month period prior to the initiation of first line axitinib in combination with IO therapy.
²Age was calculated at the index date. As only birth month and year were collected, the 15^{th} of the month was used as a proxy for patient's birthdate when calculating age.
³More than one category may have been reported.
⁴Other races include Asian, American Indian/Alaska Native, and Native Hawaiian/Pacific Islander.
⁵IMDC prognostic risk scores were provided for 157 patients and computed for 52 patients by adding prognostic risk factor information to calculate the score.

Incidence of any AEs varied by type: 48% fatigue, 38% diarrhea, 29% nausea, 22% hypertension, and 11% PPE. Median time from first-line initiation to AE onset was 1 month. Out of 729 total AEs: 376 (52%), 242 (33%), and 102 (14%), were classified as mild, moderate, and severe AEs, respectively (as defined by the Common Terminology Criteria for Adverse Events [CTCAE] v5); 130 (18%) were classified as serious AEs; and 12 (2%) were recurrent AEs (i.e., the second or later distinct episode of AE of the same type for a given patient). AEs among patients with aRCC treated with first line axitinib+IO therapy, by type of AE, are shown in Tables 6 and 7 below, which together provide the full data set.

TABLE 6
AEs among patients with aRCC treated with first line axitinib +
IO therapy, by type of AE (continued on Table 7)

| | Adverse Event | |
|---|---|---|
| Overall | Fatigue | Diarrhea |
| N - 729 | N - 234 | N - 186 |

Time from index to AE incidence (months)

| Mean ± SD | 1.5 ± 1.6 | | 1.6 ± 1.8 | | 1.4 ± 1.6 | |
|---|---|---|---|---|---|---|
| Median (IQR) | 1.0 | (0.3, 2.0) | 1.1 | (0.4, 2.0) | 1.0 | (0.3, 1.8) |

AE severity, n (%)

| | | | | | | |
|---|---|---|---|---|---|---|
| Mild | 376 | (51.6) | 139 | (59.4) | 87 | (46.8) |
| Moderate | 242 | (33.2) | 69 | (29.5) | 64 | (34.4) |
| Severe | 102 | (14.0) | 21 | (9.0) | 34 | (18.3) |
| Unknown | 9 | (1.2) | 5 | (2.1) | 1 | (0.5) |

Serious AE, n (%)

| | | | | | | |
|---|---|---|---|---|---|---|
| Yes | 130 | (17.8) | 21 | (9.0) | 51 | (27.4) |
| No | 572 | (78.5) | 203 | (86.8) | 128 | (68.8) |
| Unknown | 27 | (3.7) | 10 | (4.3) | 10 | (4.3) |
| Recurrent AE¹, n (%) | 12 | (1.6) | 2 | (0.9) | 2 | (1.1) |

¹Recurrent AEs were defined as two or more distince episodes of AE of the same type

TABLE 7
AEs among patients with aRCC treated with first line axitinib +
IO therapy, by type of AE (continued from Table 7)

| Adverse Event | | |
|---|---|---|
| Nausea | Hypertension | PPE |
| N - 146 | N - 108 | N - 55 |

Time from index to AE incidence (months)

| Mean ± SD | 1.4 ± 1.7 | | 1.6 ± 1.3 | | 1.4 ± 1.2 | |
|---|---|---|---|---|---|---|
| Median (IQR) | 0.9 | (0.2, 1.9) | 1.1 | (0.5, 2.6) | 1.0 | (0.4, 1.8) |

AE severity, n (%)

| | | | | | | |
|---|---|---|---|---|---|---|
| Mild | 84 | (57.5) | 34 | ( ) | 32 | (58.2) |
| Moderate | 44 | (30.1) | 48 | ( ) | 17 | (30.9) |
| Severe | 18 | (12.3) | 23 | ( ) | 6 | (10.9) |
| Unknown | 0 | (0.0) | 3 | ( ) | 0 | (0.0) |

Serious AE, n (%)

| | | | | | | |
|---|---|---|---|---|---|---|
| Yes | 17 | (11.6) | 28 | ( ) | 13 | (23.6) |
| No | 125 | (85.6) | 75 | ( ) | 41 | (74.5) |
| Unknown | 4 | (2.7) | 5 | ( ) | 1 | (1.8) |
| Recurrent AE¹, n (%) | 5 | (3.4) | 2 | ( ) | 1 | (1.8) |

¹Recurrent AEs were defined as two or more distince episodes of AE of the same type

AE management strategies are shown in Table 8 below and were defined as follows:
No action indicates that no modifications to axitinib and/or IO treatment and no supportive care were reported to address the AE.
Axitinib modifications include AEs managed via axitinib dose reduction and/or temporary treatment interruption (with or without additional IO modifications and/or supportive care).
Supportive care includes typical treatments for each type of AE, and primarily consisted of anti-emetics, anti-diarrheals, anti-hypertensives, and topical treatments.
IO modifications include IO treatment interruptions and/or treatment discontinuation (with or without supportive care).
Axitinib discontinuation includes AEs that were ultimately managed via axitinib discontinuation, regardless of whether other management strategies (e.g., axitinib modifications) were used prior to discontinuation.

Out of 729 total AEs, 251 (34%) and 198 (27%) were managed with axitinib modifications and no action, respectively. Among severe AEs (N=102), 32 (31%) and 15 (15%) were managed with axitinib modifications and no action, respectively.

Of 251 AEs managed with axitinib modifications, 60% dose reduced and 49% stopped temporarily. Of 32 severe AEs managed with axitinib modifications, 47% dose reduced and 59% stopped temporarily.

TABLE 8
Management Strategies for AEs Experienced Among Patients with aRCC
Treated with First Line Axitinib + IO Therapy, by AE Severity

| | | | AE Severity¹ | | | | | |
|---|---|---|---|---|---|---|---|---|
| | Overall | | Mild | | Moderate | | Severe | |
| | N - 729 | | N - 376 | | N - 242 | | N - 102 | |
| No action,² n (%) | 198 | (27.2) | 131 | (34.8) | 43 | (17.8) | 15 | (14.7) |
| Axitinib modifications,³ n (%) | 251 | (34.4) | 87 | (23.1) | 132 | (54.5) | 32 | (31.4) |
| Dosage reduction | 150 | (59.8) | 50 | (57.5) | 85 | (64.4) | 15 | (46.9) |
| Treatment interruption | 124 | (49.4) | 41 | (47.1) | 64 | (48.5) | 19 | (59.4) |
| Axitinib modifications with IO | 96 | (38.2) | 38 | (43.7) | 48 | (36.4) | 10 | (31.3) |
| modification,³ n (%) | | | | | | | | |
| Axitinib dosage reduction + IO | 43 | (44.8) | 18 | (47.4) | 22 | (45.8) | 3 | (30.0) |
| modification | | | | | | | | |
| Axitinib treatment interruption + | 70 | (72.9) | 23 | (60.5) | 38 | (79.2) | 9 | (90.0) |
| IO modification | | | | | | | | |
| Supportive care only,⁴ n(%) | 202 | (27.7) | 151 | (40.2) | 45 | (18.6) | 6 | (5.9) |
| IO modification only (with or | 24 | (3.3) | 7 | (1.9) | 12 | (5.0) | 5 | (4.9) |
| without supportive care),³ n (%) | | | | | | | | |
| Axitinib discontinuation,⁵ n (%) | 54 | (7.4) | 0 | (0.0) | 10 | (4.1) | 44 | (43.1) |

¹There were 9 AEs for which the severity was unknown. AE severity was defined by the CTCAE version 5 for each AE type.
²No action indicates that no modifications to axitinib, no modifications to IO therapy, and no supportive care were reported to address the AE.
³Axitinib modifications consist of axitinib dose reductions and/or treatment interruptions. IO modifications consistent of IO treatment interruptions and/or treatment discontinuation.
⁴Primarily consists of anti-emetics, anti-diarrheals, anti-hypertensives, and topical treatments.
⁵This category also includes AEs that were managed with other management strategies listed above (e.g., axitinib modifications) before axitinib discontinuation.

The comparison of AE resolution/improvement and time to resolution/improvement among AEs treated with axitinib modifications versus no action is shown in Tables 9 and 10 below and summarized as follow:
A significantly higher proportion of AEs treated with axitinib modification were reported as resolved/improved as the final disposition, relative to AEs where no action was taken, both across all AEs (84% vs. 60%; p<0.001) and among severe AEs (81% vs. 7%; p<0.001).
Among AEs with reported dates of resolution/improvement, median time to resolution/improvement (from AE onset) was numerically higher for AEs treated with axitinib modification vs. no action, both across all AEs (18 vs. 31 days) and among severe AEs (16 vs. 53 days).
Among AEs that resolved/improved, median time from AE management strategy initiation to resolution/improvement was 15 days for all AEs and 13 days for severe AEs treated with axitinib modification.

TABLE 9
Comparison of AE Resolution/Improvement
and Time to Resolution/Improvement among AEs Treated
with Axitinib Modifications Versus No Action Across All AEs

| | All AEs (N = 449) | | | | |
|---|---|---|---|---|---|
| | | | Axitinib | | |
| | No action¹ | | modification² | | |
| | 198 (44%) | | 251 (56%) | | P-value |
| Resolution/improvement³, n (%) | 111 | (60%) | 208 | (84%) | <0.001 |

Days to resolution/improvement,⁴ median (IQR)

| | | | | |
|---|---|---|---|---|
| From AE onset | 31 | (15, 62) | 18 | (10, 29) |

| | | | |
|---|---|---|---|
| From strategy initiation | — | 15 | (8, 28) |

¹Indicates that no interventions of any type were given.
²Includes axitinib dose reduction and/or treatment interruption, with or without any IO modifications.
³proportions are among AEs with known outcome. P-values were calculated using Chi-squared test for all AEs and Fisher's exact test for severe AEs (as the latter included categories with <5 patients.
⁴assessed where dates AE resolution/improvement were available.

TABLE 10
Comparison of AE Resolution/Improvement
and Time to Resolution/Improvement among AEs Treated
with Axitinib Modifications Versus No Action Across Severe AEs

| | Sever AEs (N = 47) | | | | |
|---|---|---|---|---|---|
| | | | Axitinib | | |
| | No action¹ | | modification² | | |
| | 15 (32%) | | 32 (68%) | | P-value |
| Resolution/improvement³, n (%) | 1 | (7%) | 26 | (81%) | <0.001 |

Days to resolution/improvement,⁴ median (IQR)

| | | | | |
|---|---|---|---|---|
| From AE onset | 53 | (53, 53) | 16 | (6, 29) |

| | | | |
|---|---|---|---|
| From strategy initiation | — | 13 | (5, 27) |

¹Indicates that no interventions of any type were given.
²Includes axitinib dose reduction and/or treatment interruption, with or without any IO modifications.
³proportions are among AEs with known outcome. P-values were calculated using Chi-squared test for all AEs and Fisher's exact test for severe AEs (as the latter included categories with <5 patients.
⁴assessed where dates AE resolution/improvement were available.

Limitations

With an analysis of non-randomized AE management strategy groups, unmeasured confounding and potential biases (e.g., selection bias) could account for observed differences in AE resolution/improvement outcomes.

In contrast to clinical trials with protocol-specified definitions of clinical events, assessments of AE outcomes in retrospective studies of real-world clinical practice may not be made consistently across patients and across physician practices.

Missing data may exist, as AEs never reported to a physician (i.e., if they were self-resolved without seeking care) will not be captured. Moreover, actions taken to manage AEs (e.g., axitinib dose reductions) may be underreported if they were not consistently reported by physicians. As such, documented management strategies for AEs would only apply to AEs that require healthcare intervention.

Conclusions

Patients whose AEs were managed with axitinib treatment modifications, including dose reduction and/or treatment interruption, had numerically higher AE resolution/improvement rates and shorter time to resolution/improvement compared to no action, both across all AEs and among severe AEs. Despite potential physician responder bias, this real-world study highlighted the importance of proactive therapy management strategies to enable optimal treatment with axitinib+IO combination treatment.

All references cited herein, including patent applications, patent publications, and UniProtKB/Swiss-Prot Accession numbers cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method of managing an adverse event in a renal cell carcinoma (RCC) patient undergoing treatment with axitinib, or a pharmaceutically acceptable salt thereof, wherein said method comprises interrupting axitinib, or a pharmaceutically acceptable salt thereof, treatment for at least 1-7 days to allow the adverse event to resolve before restarting treatment.

2. The method of claim 1, wherein the adverse event is diarrhea, hypertension, nausea, or palmar-plantar erythrodysesthesia syndrome.

3. The method of claim 2, wherein the treatment with axitinib, or a pharmaceutically acceptable salt thereof, is interrupted for 1-3 days.

4. The method of claim 2, wherein the adverse event is Grade adverse event.

5. The method of claim 4, wherein the treatment with axitinib, or a pharmaceutically acceptable salt thereof, is interrupted for 2-4 days.

6. The method of claim 1, wherein the adverse event is fatigue and said method comprises interrupting treatment for at least 4-16 days.

7. The method of claim 6, wherein the treatment is interrupted for 8 days.

8. The method of claim 1, further comprising considering reducing the dose of axitinib, or a pharmaceutically acceptable salt thereof, when restarting treatment as per recommended dose modification guidelines.

9. The method of claim 1, further comprising reducing the dose of axitinib, or pharmaceutically acceptable salt thereof, when restarting treatment as per recommended dose modification guidelines.

10. The method of claim 1, wherein the RCC patent is an advanced RCC patient.

11. The method of claim 10, wherein the advanced RCC patient is a first-line advanced RCC patient.

12. The method of claim 10, wherein the advanced RCC patient is a second-line advanced RCC patient.

13. A method of managing an adverse event in an RCC patient undergoing treatment with a combination of axitinib, or a pharmaceutically acceptable salt thereof, and an immune-oncology (IO) agent, wherein said method comprises interrupting axitinib, or a pharmaceutically acceptable salt thereof, treatment for at least 4-11 days to allow the adverse event to resolve before restarting axitinib, or a pharmaceutically acceptable salt thereof, treatment.

14. The method of claim 13, wherein the adverse event is diarrhea, fatigue, hypertension, nausea, or palmar-plantar erythrodysthesia.

15. The method of claim 13, wherein the adverse event is Grade adverse event.

16. The method of claim 13, wherein the IO agent is a programmed cell death protein 1 (PD-1) antagonist or a programmed cell death ligand 1 (PD-L1) antagonist.

17. The method of claim 16, wherein the IO agent is pembrolizumab.

18. The method of claim 16, wherein the IO agent is avelumab.

19. The method of claim 13, further comprising considering reducing the dose of axitinib, or a pharmaceutically acceptable salt thereof, when restarting treatment as per recommended dose modification guidelines.

20. The method of claim 13, further comprising reducing the dose of axitinib, or pharmaceutically acceptable salt thereof, when restarting treatment as per recommended dose modification guidelines.

21. The method of claim 13, wherein the RCC patent is an advanced RCC patient.

22. The method of claim 21, wherein the advanced RCC patient is a first-line advanced RCC patient.

23. The method of claim 21, wherein the advanced RCC patient is a second-line advanced RCC patient.
